(19) United States
(12) Patent Application Publication
(10) Pub. No.: US 2017/0360841 A1
(43) Pub. Date: Dec. 21, 2017

(54) METHODS FOR PRODUCING DOPAMINERGIC NEURONS AND USES THEREOF

(71) Applicant: UNIVERSITÉ LAVAL, Quebec (CA)

(72) Inventors: Martin Lévesque, Quebec (CA); Audrey Chabrat, Quebec (CA)

(21) Appl. No.: 15/694,451

(22) Filed: Sep. 1, 2017

Related U.S. Application Data
(62) Division of application No. 14/720,010, filed on May 22, 2015.
(60) Provisional application No. 62/002,416, filed on May 23, 2014.

(51) Int. Cl. A61K 35/30; C12N 15/113

(52) U.S. Cl. CPC A61K 35/30; C12N 15/1138; C12N 2310/531; C12N 2310/14

(57) ABSTRACT

The present application relates to a stem cell, a precursor or progenitor cell in which Plexin C1 has been inactivated, a dopaminergic (DA) neuron in which Plexin C1 has been inactivated, methods of preparing same and uses thereof for the treatment of Parkinson's disease.

### CROSS REFERENCE TO RELATED APPLICATIONS

This application is a divisional application of U.S. application Ser. No. 14/720,010, filed on May 22, 2015; which claims the benefit of U.S. provisional application Ser. No. 62/002,416, filed on May 23, 2014, the entire contents of each of which are hereby incorporated herein by reference.

### BACKGROUND

Stem cells are distinguished from other cell types by two important characteristics. First, they are unspecialized cells capable of renewing themselves through cell division, sometimes after long periods of inactivity. Second, under certain physiologic or experimental conditions, they can be induced to become tissue- or organ-specific cells with special functions. In some organs, such as the gut and bone marrow, stem cells regularly divide to repair and replace damaged tissues. In other organs, however, such as the pancreas and the heart, stem cells only divide under special conditions.

During embryonic development, stem cells form the tissues of the body from three major cell populations: ectoderm, mesoderm and definitive endoderm. Mesoderm gives rise to blood cells, endothelial cells, cardiac and skeletal muscle, and adipocytes. Definitive endoderm generates liver, pancreas and lung. Ectoderm gives rise to the nervous system, skin and adrenal tissues.

A potential application of stem cells is making cells and tissues for medical therapies. Today, donated organs and tissues are often used to replace those that are diseased or destroyed. Unfortunately, the number of people needing a transplant far exceeds the number of organs available for transplantation. Stem cells offer the possibility of a renewable source of replacement cells and tissues to treat a myriad of diseases, conditions, and disabilities including Parkinson's disease, amyotrophic lateral sclerosis, spinal cord injury, burns, heart disease, diabetes, and arthritis.

Parkinson's disease (PD) is a neurological disorder caused by a progressive degeneration of midbrain dopamine (mDA) neurons in the substantia nigra pars compacta (SNpc). The degeneration of mDA neurons causes a gradual dysfunction of the motor system leading to symptoms such as tremor, rigidity, and bradykinesia, among others. There is currently no cure for PD and, although treatments such as deep brain stimulation and levodopa can alleviate some of the symptoms, they tend to lose efficacy over time. However, the localized nature of the loss of mDA neurons in the substantia nigra (SN) makes cell replacement therapy an attractive approach to treating Parkinson's disease (PD) patients.

Clinical attempts to replace mDA neurons by transplantation of fetal midbrain tissue failed to meet the primary endpoint and were complicated by the development of off-medication dyskinesia. Both the limited clinical response and off-medication dyskinesia may be related to incomplete dopaminergic reinnervation of the striatum. Both these limitations might be resolved by transplantation of more dopaminergic neurons. Stem cells represent a promising therapeutic avenue, offering the opportunity to generate large numbers of standardized dopamine neurons for transplantation. Dopamine neurons from SNpc and VTA have totally different target sites and exhibit differential vulnerability to degeneration in Parkinson's disease. In fact, VTA neurons are relatively spared, whereas dopamine neurons from SNpc show a progressive degeneration during the progression of the disease, leading to a depletion of dopamine in the striatum (target region of SNpc neurons). A major factor limiting success in transplantation studies is incomplete reinnervation of grafted neurons. In order to develop successful stem cell-based therapies for PD, it is critical to be able to generate a homogeneous population of functional DA neurons that express the right axon guidance machinery to reach and innervate their natural targets.

### SUMMARY

The present description is based on the findings that dopaminergic axon guidance is regulated by Plexin C1, and the presence of Plexin C1 induces a misguidance of the dopaminergic neuron of the SNpc. The presence of Plexin C1 makes the neuron responsive to semaphorin 7A and causes the neuron to grow away from the dorsal striatal region.

In one aspect, there is provided a stem cell, a precursor or progenitor cell in which Plexin C1 has been inactivated.

In one aspect, the present description relates to a dopaminergic (DA) neuron in which Plexin C1 has been inactivated.

In one aspect, the present description relates to a DA neuron differentiated from a stem cell as defined herein.

In one aspect, the present description relates to the use of a DA neuron as defined herein for the treatment of Parkinson's disease in a patient in need of such treatment.

In one aspect, the present description relates to a method for the treatment of Parkinson's disease comprising administering DA neurons as defined herein to a patient in need of such treatment.

In one aspect, the present description relates to a method for cell replacement therapy in a Parkinson's disease patient comprising transplanting DA neurons as defined herein in the brain of a Parkinson's disease patient.

In one aspect, the present description relates to the use of a DA neuron as defined herein for cell replacement therapy in a Parkinson's disease patient.

In one aspect, the present description relates to the use of a stem cell as defined herein to obtain an axon guidable DA neuron.

In one aspect, the present description relates to the use of a stem cell as defined herein for the treatment of Parkinson's disease in a patient in need of such treatment.

In one aspect, the present description relates to a method for the treatment of Parkinson's disease comprising administering the use of a stem cell as defined herein to a patient in need of such treatment.

In one aspect, the present description relates to a method for cell replacement therapy in a Parkinson's disease patient comprising transplanting the use of a stem cell as defined herein in the brain of a Parkinson's disease patient.

In one aspect, the present description relates to the use of a stem cell as defined herein for cell replacement therapy in a Parkinson's disease patient.

In one aspect, the present description relates to a method for generating a guidable DA neuron, the method comprising the step of:
differentiating a stem cell as defined herein;
selecting cells having dopaminergic neuron cell markers.

In some aspects, the present description relates to the following items:
(1) A stem cell, a precursor or progenitor cell in which Plexin C1 has been inactivated.
(2) The cell of (1) wherein the stem cell is a neural stem cell or an induced pluripotent stem cell.
(3) The cell of (1) or (2), wherein Plexin C1 is inactivated by gene knock-down.
(4) The cell of (1) or (2), wherein Plexin C1 is inactivated by small interfering RNA (siRNA) or short hairpin RNA (shRNA).
(5) The cell of (1) or (2), wherein Plexin C1 is inactivated by gene knock-out.
(6) The cell of (1) or (2), wherein Plexin C1 is inactivated by using a Cre-Lox recombination system.
(7) The cell of (1) or (2), wherein Plexin C1 is inactivated by using a Zinc finger system.
(8) The cell of (1) or (2), wherein Plexin C1 is inactivated by using a CRISPR/Cas system.
(9) A dopaminergic (DA) neuron in which Plexin C1 has been inactivated.
(10) The DA neuron of (9), wherein the DA neuron is human.
(11) The DA neuron of (9) or (10), wherein the neuron is a midbrain neuron.
(12) The DA neuron of (9), (10) or (11), wherein Plexin C1 is inactivated by gene knock-down.
(13) The DA neuron of (9), (10) or (11), wherein Plexin C1 is inactivated by small interfering RNA (siRNA) or short hairpin RNA (shRNA).
(14) The DA neuron of (9), (10) or (11), wherein Plexin C1 is inactivated by gene knock-out.
(15) The DA neuron of (9), (10) or (11), wherein Plexin C1 is inactivated by using a Cre-Lox recombination system.
(16) The DA neuron of (9), (10) or (11), wherein Plexin C1 is inactivated by using a Zinc finger system.
(17) The DA neuron of (9), (10) or (11), wherein Plexin C1 is inactivated by using a CRISPR/Cas system.
(18) A DA neuron differentiated from a cell as defined in any one of (1) to (8).
(19) The DA neuron of (18), wherein the DA neuron is human.
(20) The DA neuron of (18) or (19), wherein the neuron is a midbrain neuron.
(21) Use of a DA neuron as defined in any one of (9) to (19) for the treatment of Parkinson's disease in a patient in need of such treatment.
(22) A method for the treatment of Parkinson's disease comprising administering DA neurons as defined in any one of (9) to (19) in a patient in need of such treatment.
(23) A method for cell replacement therapy in a Parkinson's disease patient comprising transplanting DA neurons as defined in any one of (9) to (19) in the brain of a Parkinson's disease patient.
(24) Use of a DA neuron as defined in any one of (9) to (19) for cell replacement therapy in a Parkinson's disease patient.
(25) Use of a stem cell as defined in any one of (1) to (8) to obtain an axon guidable DA neuron.
(26) A method for generating a guidable DA neuron, the method comprising the step of: a) differentiating a stem cell as defined in any one of (1) to (8); b) selecting cells having dopaminergic neuron cell markers.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1. Phenotype analysis of Lmx1a and Lmx1b double conditional mutant mice at postnatal day 1. Double conditional mutant mice for Lmx1a/b fail to innervate dorsal striatum (B, F). However, the distribution and the number of mDA neurons in the midbrain are not different from controls (C-E), suggesting that Lmx1a/b regulate mDA axons development. G, Double immunolabeling using two dopamine neuron markers, TH and DAT, confirms axonal targeting defects in Lmx1a/b conditional mutants.

FIG. 2. Plexin C1 expression is up-regulated in Lmx1a/b mutant mDA neurons, indicating that Lmx1a/b play a repressive role on this gene. (A) Schematic showing the protocol followed to identify Lmx1a/b target genes at E15.5. (B-C) Validation of Plexin C1 by qPCR (B) and in situ hybridization (C). In situ hybridization also shows an expansion of Plexin C1 in SNpc neurons of both Lmx1a/b double mutant (C) and double conditional mutants embryos (D).

FIG. 3. Tracing experiments showing aberrant dopaminergic axonal connections in Lmx1a/b conditional mutants. In control animals, Flurorogold™ (retrograde tracer) injections in Nucleus accumbens label mDA neurons in VTA, but similar injections in Lmx1a/b conditional mutants result in labeling both VTA and SNpc neurons.

FIG. 4(A-C). Genes regulatory network model for nigrostriatal and mesolimbic axons development. Based on our data, Plexin C1 expression is negatively regulated by Lmx1a and b, while Otx2, expressed by VTA neurons, positively controls Plexin C1. In mutant mice lacking both Lmx1a and b, Plexin C1 expression is expanded to all mDA neurons. This aberrant expression by SNc neurons makes them responsive to Sema7a (expressed in dorsal striatal region) and causes their axons to grow away from this repellent cue. (C) Expression of Sema7a on a mouse brain sagittal section. Asterisk (*) in C indicates dorsal striatal region rich in Sema7a.

FIG. 5. In vitro experiments demonstrating the effect of Sema7a on mDA axons. (A-D) Bath application of Sema7a causes mDA growth cones enlargement and affect axonal length. Explants were incubated with an antibody directed against β1-integrin; thus Sema7a would bind only to its other receptor: Plexin C1. Embryonic ventral midbrain explants from E14.5 Pitx3-GFP embryos were grown for 48 h in collagen matrix (Matrigel, BD), then exposed to Sema7a (0.5 ug/ml) for 2 h prior to fixation. Sema7a seems to induce phosphorylation of Src kinases in mDA growth cones (B). (E) Culture of mDA explants near HEK cells transfected to express Sema7A seems to show a repulsive effect for axons growing away from cells releasing Sema7a. These experiments demonstrate a chemo-repulsive effect of Sema7a on axon guidance.

FIG. 6: Sema7a application induces growth cone stalling and collapsing. (A) representation of a novel technique to study mDA axon guidance under multiphotons microscope. The schematic shows sagittal brain section from a Pitx3-GFP mouse embryo at E14.5. A puff application of Sema7a creates a gradient. Time lapse imaging is then performed every 5 min to monitor growth cones movements. (B) Example of midbrain dopaminegic growth cone imaged for more than an hour without sign of photobleaching or damages. (C) Example of mDA growth cone reaction to Sema7a application. In this example, Sema7a induces growth cone stalling and collapsing. Images were acquired every 5 min for 25 min prior Sema7a application.

FIG. 7. Sema7a KO mice analysis. (A-C) Plexin C1 immunopositive mDA axons are more numerous in dorsal striatal region of Sema7a KO mice compared to control animals. These data suggest that lack of Sema7a chemorepulsive cue induces dopaminergic axon guidance defects. (D-E) Li-Cor quantification of dopaminergic innervation in Sema7a KO demonstrates an overall increase in TH+ axons in striatal regions, suggestion that Sema7a regulates mDA axonal arborisation. (F) Stereological neuronal counting did not reveals any significant difference in the number of mDA neuron number. (G-I) Quantification of Plexin C1 expression using optical density measurement. More Plexin C1 signal is observed in the dorsal part of the striatum for the mutant, as previously show in A-C.

### DETAILED DESCRIPTION

In one aspect, the present description relates to an enriched stem cell population, precursor or progenitor cell population in which Plexin C1 has been inactivated.

In one aspect, the present description relates to an enriched dopaminergic (DA) neuron population in which Plexin C1 has been inactivated.

In one aspect, the present description relates to a method to derive or differentiate DA neurons from stem cells in which Plexin C1 has been inactivated.

In one aspect, the present description relates to a method of directly converting cells into DA neurons in which Plexin C1 has been inactivated.

In one aspect, DA neurons may be generated from direct conversion of other cell types (e.g. fibroblasts) and where Plexin C1 is inactivated.

In one aspect, the present description relates to a method to obtain DA neurons in which Plexin C1 is inactivated and that could be guided to the dorsal striatal region of the brain.

In one aspect, the Plexin C1 inactivated DA neurons are not repelled from the dorsal striatal region of the brain by semaphorin 7a (Sema7A).

In one aspect, the DA neurons are midbrain dopamine neurons (mDA neurons).

In one aspect, the Plexin C1 inactivated DA neurons (e.g. mDA neurons) may be used in cell replacement therapy for the treatment of Parkinson's disease (PD).

In one aspect the, the Plexin C1 inactivated DA neurons (e.g. mDA neurons) may be used to regenerate a functional network in a PD patient.

In one aspect, the DA neuron is human.

In one aspect, the DA neuron is a midbrain neuron.

### Inactivation of Plexin C1

Plexin C1 (PLXNC1 gene; PLXC1 protein, CD232, VESPR) is a receptor for the GPI-anchored semaphorin (Sema7A). Plexin C1 is described in Tamagashi et al. (Cell. 1999 Oct. 1; 99(1):71-80). Inactivated Plexin C1 means that the activity and/or expression of Plexin C1 is significantly reduced in the cell relative to a control. Methods for inactivating gene expression or activity are well known in the art. In one aspect, Plexin C1 can be inactivated with inhibitors, antibodies, gene knock-out or gene knock-down. For example, Plexin C1 could be inactivated by using well known techniques such as siRNA, shRNA, Cre-Lox recombination systems, Zinc finger systems or CRISPR/Cas systems (e.g. CRISPR/Cas9).

In one aspect, neurons in which Plexin C1 has been inactivated will not be repelled from the dorsal striatal region of the brain by Sema7a. Methods for determining gene expression and protein activity are well known in the art.

In one aspect, an axon guidable DA neuron is a DA that will not be repelled from the dorsal striatal region of the brain by Sema7a.

In one aspect, neurons in which Plexin C1 has been inactivated will not be repelled from the dorsal striatal region of the brain by Sema7a. Methods for determining gene expression and protein activity are well known in the art.

In the methods of the present description, Plexin C1 may be inactivated at any point involved in the generation of the inactivated Plexin C1 DA neurons.

### Cell Replacement Therapy and Methods for Deriving and Differentiating DA Neurons

Cell therapy or transplantation refers to the injection or administration of cells to a subject in order to treat a specific condition. In one aspect, cell replacement therapy generally consists in introducing newly generated cells in accordance with the present description to compensate a lack of functional cells. In one aspect, the cells in accordance with the present description can be implanted in the striatum, where the loss of innervation is observed, or in the midbrain to replace mDA neurons from SNpc which degenerated during PD progression.

In one aspect, the present description relates to the use of DA neurons to replace lost dopaminergic neurons in Parkinson's disease (PD).

Several sources of cells could be used for cell replacement therapy in the treatment of PD (see for example; Meyer et al. 2010,).

### Sources of Stem Cells for Cell Replacement Therapy in PD

Embryonic Stem Cells (ES Cells)

ES cells were the first cells used for cell-replacement therapy because of their properties of self-renewing, pluripotency, and capacity to differentiate into a specific cell type. ES cells can be obtained by isolation from the inner mass of a blastocyst. Autologous embryonic stem cells can be generated through therapeutic cloning. ES cells can be differentiated into functional DA neurons

Neural Stem Cells (NSCs)

NSCs are multipotent cells having the capacity to self-renew, but unlike ES cells, NSCs are less likely to induce tumorigenicity. NSCs can differentiate into the main cell-types of the brain: neurons, oligodendrocytes, and astrocytes.

Fetal NSCs

Fetal NSCs are multipotent stem cells, which seem to induce only mild immunoreactions, but no tumor formation.

Adult NSCs

Adult NSCs are autologous cells and are present in two regions of the adult mammalian brain known as neurogenic regions: the hippocampus, and the subventricular zone (SVZ) of the lateral ventricles. These multipotent cells are able to differentiate into neural cells.

Adult Multipotent Stem Cells

Adult multipotent stem cells, as iPSCs and adult NSCs, are patient-specific. Several sources of these cells are available like: umbilical cord blood, bone marrow, adipose-derived, placental and amniotic fluid.

Hematopoietic stem cells (HSCs), and mesenchymal stem cells (MSCs) can be isolated from the human umbilical cord blood (UCB), and represent an important pool of cells for cell transplantation. These UCB stem cells are able to differentiate into neurons using pro-neurogenic factors.

Bone marrow stromal cells, also known as mesenchymal stem cells (MSCs), represent another source of adult multipotent stem cells. These cells are able to become a source of neural stem cells (NSCs) by bringing the good factors for their conversion.

Induced Pluripotent Stem Cells (iPSCs)

iPSCs can also be a source of stem cells (Takahashi and Yamanaka, 2006; Takahashi et al., 2007). These cells have the capacity to self-renew and can be differentiated into various cell types (Gao et al., 2013). iPSCs can be obtained by therapeutic cloning. This therapeutic cloning consists in reprogramming adult cells, like fibroblasts for example, into ES cells by nuclear transfer to oocytes (Meyer et al., 2010). iPSCs can also be obtained by fusion of adult cells with ES cells (Meyer et al., 2010). Added to these two techniques for reprogramming cells into pluripotent cells, another method has been developed using a limited set of transgenes including Oct3/4, Sox2, c-Myc, and Klf4 (Takahashi and Yamanaka, 2006). These three different techniques for iPSCs production lead to an enough quantity to enable disease investigation and drug development (GAO et al., 2013). In 2008, Wernig and co-workers, managed to differentiate iPSCs into functional mDA neurons. Indeed, the good function of these newly generated mDA neurons was observed by an improvement of parkinsonian symptoms after implantation of these cells in the striatum of rats (Wernig et al., 2008).

In order to avoid the tumorigenic effect observed with iPSCs replacement therapy, improvements in techniques to generate them were performed. Actually, human somatic cells can be reverted into pluripotent cells using another set of transgenes including Oct4, Sox2, Nanog, and Lin28, without c-Myc possible factor in the induction of tumorigenicity (Yu et al., 2007). These transgenes can be delivered using different methods: retroviral transduction, plasmid transfection, direct reprogramming factor delivery, and mRNA transfection (GAO et al., 2013). Having the choice between these four methods of transgenes delivery is interesting, because even if retroviral transduction is a very effective way to deliver transgenes, retroviral vectors might induce tumorigenicity by integration of the viral DNA into the genome of transduced cells (Okita et al., 2008).

Moreover, in a publication of Doi and co-workers from March 2014, a new technique to avoid tumorigenic content or inappropriate cells transplantation when using iPSC-derived donor cells for cell replacement therapy has been presented. They have shown an efficient way to isolate specifically human iPSC-derived DA progenitor cells by cell sorting using a floor plate marker, CORIN (Doi et al., 2014). The transplantation of these iPSCS into 6-OHDA-lesioned rats, have shown a survival of CORIN+ cells, and their differentiation into midbrain DA neurons in vivo. These “pure” iPSCs transplantation led to a significant improvement of the motor behavior, without tumor formation (Doi et al., 2014).

Fibroblast

Skin fibroblasts can be converted directly into DA cells, bypassing the pluripotent stage (Doi et al., 2014). Mitchell and co-workers, using only the Oct4 factor, manage to perform a direct conversion process of primary adult human fibroblasts (hFib) to neural progenitor cells (NPC). These NPCs present the same features as hNPCs. They are able to proliferate, express neural stem/progenitor markers, and to differentiate into the all three major subtypes of neural cells (Mitchell et al., 2014).

As used herein, “stem cell” defines a cell (including pluripotent stem cells) with the ability to divide for indefinite periods in culture and give rise to specialized cells. Stem cells include, for example, somatic (adult) and embryonic stem cells. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell derived from the embryo that has the potential to become a wide variety of specialized cell types. An embryonic stem cell is one that has been cultured under in vitro conditions that allow proliferation without differentiation. Non-limiting examples of embryonic stem cells are the HES2 (also known as ES02) cell line available from ESI, Singapore and the H1 (also known as WA01) cell line available from WiCells, Madison, Wis. In addition, for example, there are 40 embryonic stem cell lines that are recently approved for use in NIH-funded research including CHB-1, CHB-2, CHB-3, CHB-4, CHB-5, CHB-6, CHB-8, CHB-9, CHB-10, CHB-11, CHB-12, RUES1, HUES1, HUES2, HUES3, HUES4, HUES5, HUES6, HUES7, HUES8, HUES9, HUES10, HUES11, HUES12, HUES13, HUES14, HUES15, HUES16, HUES17, HUES18, HUES19, HUES20, HUES21, HUES22, HUES23, HUES24, HUES26, HUES27, and HUES28. Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4.

An “induced pluripotent stem cell” or “iPSC” or “iPS cell” refers to an artificially derived stem cell from a non-pluripotent cell, typically an adult somatic cell, produced by inducing expression of one or more reprogramming genes or corresponding proteins or RNAs. Such stem cell specific genes include, but are not limited to, the family of octamer transcription factors, i.e. Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs and methods of preparing them are described in Doi et al. Stem Cell Reports; Vol. 2; 337-350; Mar. 11, 2014.

A neural stem cell is a cell that can be isolated from the adult central nervous systems of mammals, including humans. They have been shown to generate neurons, migrate and send out axons and dendritic projections and integrate into pre-existing neural circuits and contribute to normal brain function

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. Examples of progenitor cells include, but are not limited to, satellite cells found in muscles, intermediate progenitor cells formed in the subventricular zone, bone marrow stromal cells, periosteum progenitor cells, pancreatic progenitor cells and angioblasts or endothelial progenitor cells. Examples of progenitor cells may also include, but are not limited to, epidermal and dermal cells from neonatal organisms.

A “neural precursor cell”, “neural progenitor cell” or “NP cell” refers to a cell that has a capacity to differentiate into a neural cell or neuron. A NP cell can be an isolated NP cell, or derived from a stem cell including but not limited to an iPS cell. Neural precursor cells can be identified and isolated by neural precursor cell specific markers including, but limited to, nestin and CD133. Neural precursor cells can be isolated from animal or human tissues such as adipose tissue. Neural precursor cells can also be derived from stem cells or cell lines or neural stem cells or cell lines.

In one aspect, the stem cell is a neural stem cell or an induced pluripotent stem cell.

As used herein, “differentiation” refers to a change that occurs in cells to cause those cells to assume certain specialized functions and to lose the ability to change into certain other specialized functional units. Cells capable of differentiation may be any of totipotent, pluripotent or multipotent cells. Differentiation may be partial or complete with respect to mature adult cells.

A neuron is an electrically excitable cell that processes and transmits information through electrical and chemical signals. A chemical signal occurs via a synapse, a specialized connection with other cells. Neurons connect to each other to form neural networks. Neurons are the core components of the nervous system, which includes the brain, spinal cord, and peripheral ganglia.

Dopaminergic neurons. Dopamine is a neurotransmitter that acts on DI type (DI and D5) Gs coupled receptors, which increase cAMP and PKA, and D2 type (D2, D3, and D4) receptors, which activate Gi-coupled receptors that decrease cAMP and PKA. Dopamine regulates mood, reward behaviour and motor function. Dopamine modulates both pre and post synaptic neurotransmission. Loss of dopamine neurons in the substantia nigra has been linked to Parkinson's disease.

Methods for differentiating DA neurons are well known in the art. See for example Takahashi et al. Stem Cell Reports; Vol. 2; 337-350; Mar. 11, 2014, Burbach et al, Nature Review, Vol. 8, 2007, 21-29 and Caiazzo et al. Nature, Vol. 476, 2011, 224-229.

### Therapeutic Uses

Stem cells and/or DA neurons in accordance with the present description can be injected locally or systemically into a patient.

The expression “systemic administration” refers to a route of administration that is either enteral or parental. Enteral administration is known to involve absorption of the component through the gastrointestinal tract. Parental refers to administration outside the digestive tract and for instance intravenous injection. When the cells of the present description are enterally administered, they may be inserted into dendrimers, lipoprotein-based drug carriers polymeric or micelles as known in the art.

The expression “local administration” refers to application of the cells to a localized area of the body or to the surface of a body part. In one aspect, the DA neurons in accordance with the present description can be transplanted directly into the brain of a patient in need of such treatment (e.g. PD patient).

In one aspect, the patient is a patient diagnosed with PD. In a further aspect, the patient is a PD patient that suffers from a loss of mDA neurons in the Substantia nigra pars compacta (SNpc) region of the brain.

The present description also provides a cell therapy method for treating a patient in need thereof.

The expression “treating a patient in need thereof” refers to any person susceptible of suffering or suffering from a disease (e.g. PD) from which the symptoms could be alleviated or reduced. More specifically, the subject consists of a human.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present invention will be more readily understood by referring to the following example. The examples are illustrative of the wide range of applicability of the present description and are not intended to limit its scope. Modifications and variations can be made therein without departing from the spirit and scope of the invention. Although any methods and materials similar or equivalent to those described herein can be used in the practice for testing of the present description, the following methods and materials are described. The issued patents, published patent applications, and references that are cited herein are hereby incorporated by reference to the same extent as if each was specifically and individually indicated to be incorporated by reference. In the case of inconsistencies, the present disclosure will prevail.

### EXAMPLES

### Aberrant Dopaminergic Axonal Connections in Lmx1a/b Double Conditional Mutant

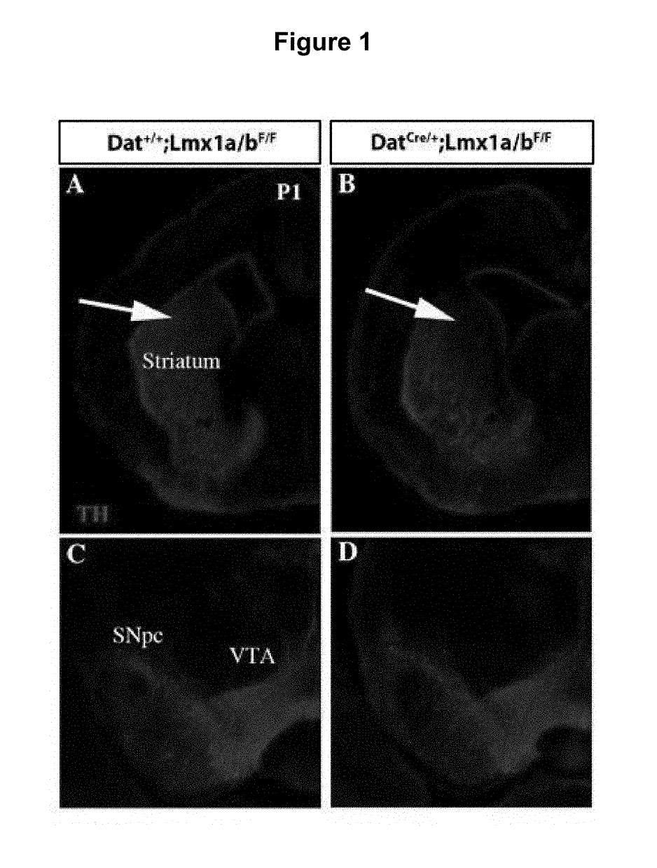

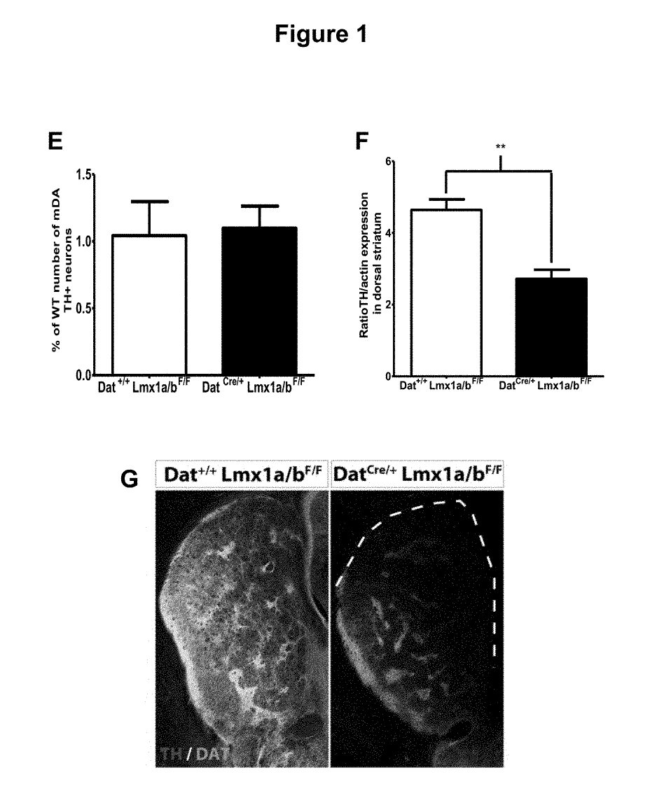

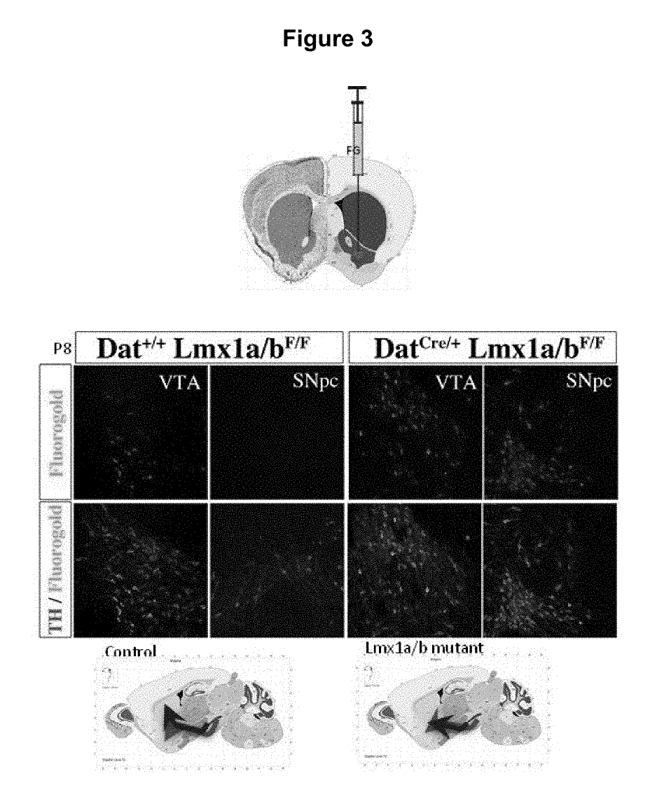

Mutant mice where Lmx1a and Lmx1b genes have been conditionally inactivated in midbrain dopaminergic (mDA) neurons show aberrant axon connections. Indeed, a severe loss of dopaminergic innervation in the dorsal part of the striatum was observed for Lmx1a/b double mutant mice (FIG. 1). This loss of innervation was detected using immunofluorescence techniques revealing tyrosine hydroxylase (TH, rate limiting enzyme for Da production) and dopamine transporter (DAT). Stereological counting of the number of mDA neurons revealed no difference between the wild type (Wt) and the mutants (FIG. 1. E). Without being bound by any specific theory, the inventors believe that this loss of innervation is due to axon misguidance, and that Lmx1a/b transcription factors regulate mDA axon targeting. To verify this hypothesis, the retrograde neuronal tracer Fluorogold™ (FG) was injected in mDA neuron target areas using stereotaxic intervention. In control animals, FG injections in nucleus accumbens labeled mDA neurons in the VTA, while similar injections in Lmx1a/b conditional mutants resulted in labeling both VTA and SNpc neurons (FIG. 3). These results confirmed the aberrant dopaminergic axonal connections in Lmx1a/b conditional mutants.

### Lmx1a/b Regulate Plexin C1 Expression

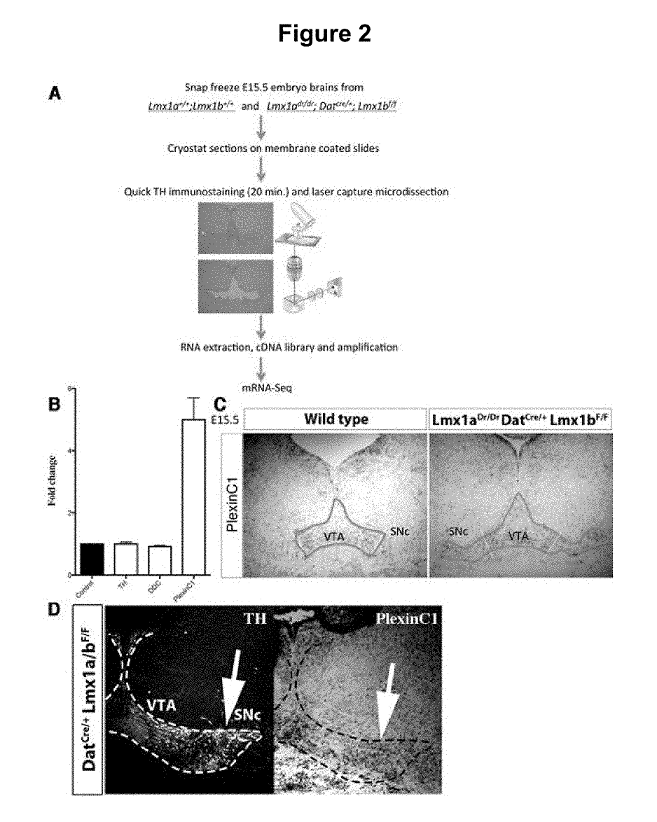

In order to identify target genes for Lmx1a/b, a gene profiling experiment (mRNA sequencing, FIG. 2) was performed to compare genes expressed by mDA neurons from mutants and controls animals. The results showed that over 500 genes were regulated by Lmx1a/b. Among them, a five-fold overexpression of the gene coding for Plexin C1 was observed in KO mice for Lmx1a/b. Plexin C1 expression is up-regulated in Lmx1a/b mutant mDA neurons, indicating that Lmx1a/b play a repressive role on this gene. Validation of Plexin C1 as a target for Lmx1a/b was done by qPCR and in situ hybridization (FIG. 2). Analysis of Plexin C1 expression by in situ hybridization revealed that Plexin C1 expression is expanded to all mDA neurons. For the controls, Plexin C1 was expressed only in VTA neurons, whereas its expression was detected in both the SNpc and the VTA for Lmx1a/b conditional mutants. Without being bound by any specific theory, the inventors believe that the aberrant expression of Plexin C1 induced a misguidance of the SNpc dopaminergic neurons. Instead of innervating their normal targeted site in the dorsal striatum, the dopaminergic neurons of the SNpc are guided to the nucleus accumbens, usually targeted by the VTA dopaminergic neurons.

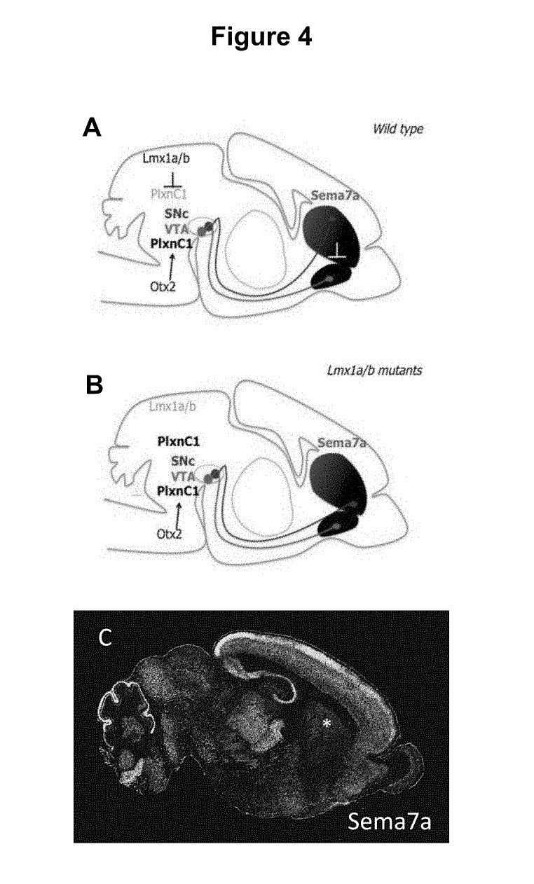

Preliminary results led to propose a model explaining the role of Lmx1a/b in the regulation of dopaminergic neurons axon guidance with genes regulatory network for nigrostriatal and mesolimbic axons development (FIG. 4). Based on our data, Plexin C1 expression is down-regulated by Lmx1a and Lmx1b, while Otx2, another transcription factor expressed by VTA neurons, positively controls Plexin C1 (Chung et al. Brain, 2010). In mutant mice lacking both Lmx1a and Lmx1b, Plexin C1 expression is expanded to all mDA neurons. This aberrant expression of Plexin C1 by SNpc neurons makes them responsive to semaphorin 7A (Sema7a), a known ligand expressed in the dorsal striatal region (FIG. 4), causing their axons to grow away from this repellent cue.

### Role of Sema7a on mDA Neurons Axon Guidance

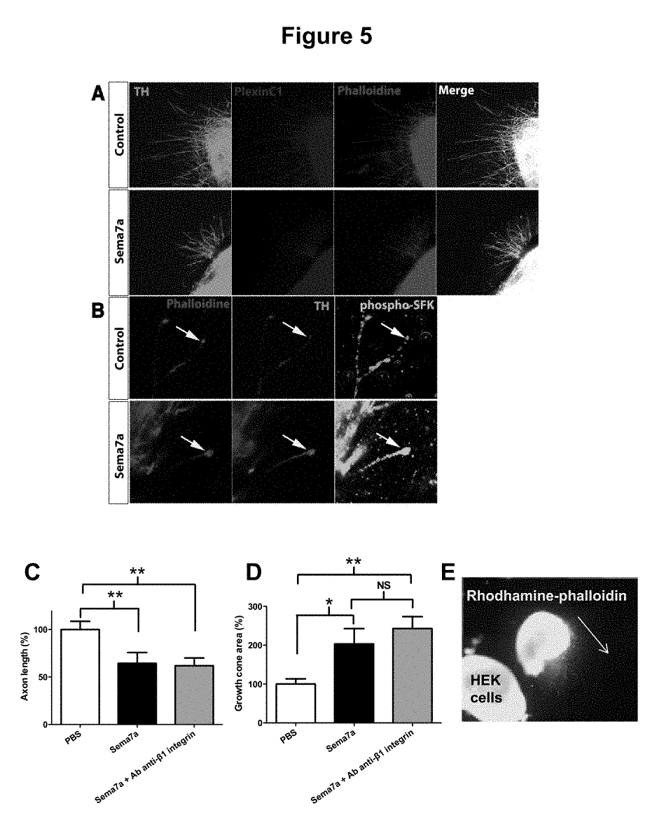

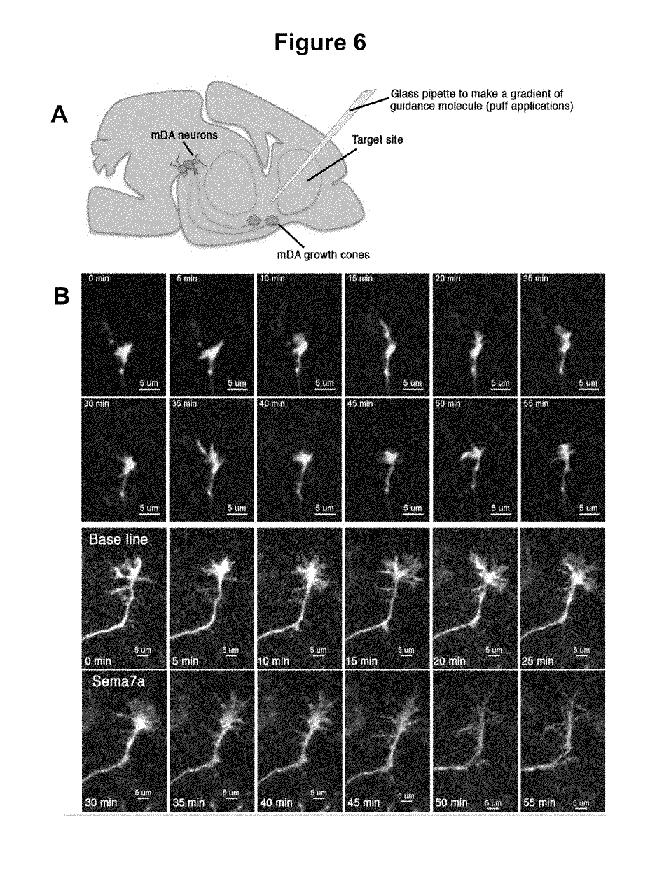

To study the effect of Sema7a on mDA neurons, in vitro experiments were performed. Embryonic ventral midbrain explants were grown for 48 h in collagen matrix (Matrigel™, BD), then exposed to Sema7a (0.5 μg/mL) in a bath application for 2 h prior fixation. The effects of Sema7a application on mDA neurons were drastic. A decrease in the axon length, and an increase of the growth cone area for explants treated with Sema7a was observed. The increase of the growth cone could be due to microtubule looping (Purro et al., 2008), a phenomenon frequently observed following bath incubation of a chemo-repulsive molecule. The decrease in axon length indicates a probable retraction of mDA axons induced by Sema7a. Because Sema7a can also bind β1-integrin, experiments where done in a presence of an antibody blocking the binding of Sema7a on β1-integrin. Results obtained using this function-blocking antibody indicate that the chemo-repulsive effect of Sema7a on mDA axons were specific to the binding of Sema7a on the Plexin C1 receptor (FIG. 5). In a second series of in vitro experiments, mDA explants were grown near HEK cells transfected to express Sema7A. Explants were incubated with an antibody directed against β1-integrin, thereby blocking the ability of Sema7a to bind thereto. These results further indicate that Sema7a induces a chemo-repulsive effect on mDA axon guidance in vitro. Finally, a novel technique to study mDA axon guidance under multiphotons microscope allowed following the effect of a gradient of Sema7a on mDA growth cones. This ex vivo slices experiment was done on living sagittal sections at E14.5 (FIG. 6). On these sagittal sections, a puff of Sema7a was performed at the level of growth cones, and time lapse imaging was performed every 5 minutes to monitor growth cone movement. These results revealed a stalling and then a collapsing of growth cones after Sema7a application, showing once more a chemo-repulsive effect of Sema7a on mDA neurons axon guidance.

### Sema7a Regulates mDA Neurons Axon Guidance and Sprouting

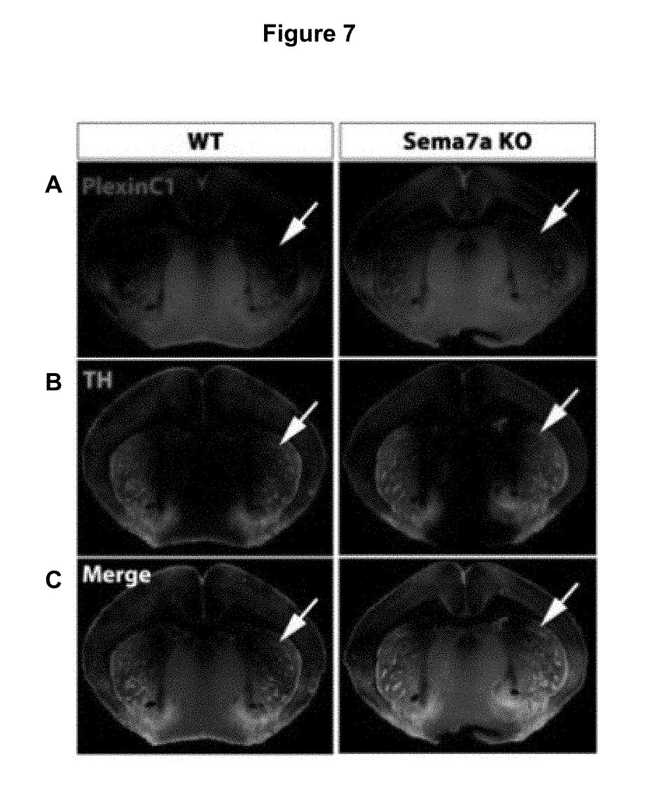

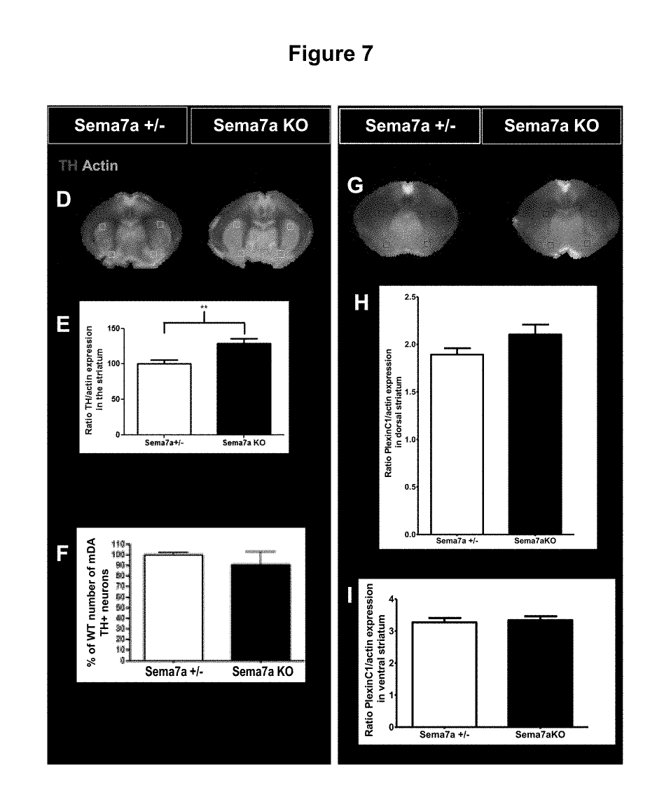

To further investigate the role of Sema7a/Plexin C1 on mDA neuron targeting, Sema7a knockout mice were analyzed. TH immunolabeling revealed an increase of dopaminergic innervation in the striatum (FIG. 7). This increase was also quantified with an optical density measurement method. In order to verify whether this increased number of TH+ axons was not due to an increase in the number of mDA neurons, stereological neuronal counting was performed. No significant difference in the number of mDA neurons between Sema7a KO and Sema7a+/− mice was observed. These data suggest that Sema7a regulates mDA axonal arborization. Immunolabeling for Plexin C1 in Sema7a KO mice also revealed an increase in mDA axons containing Plexin C1 in the dorsal part of the striatum. These axons are likely coming from VTA neurons and, due to the absence of Sema7a (chemo-repulsive cue), these mDA axons are not repelled from dorsal striatum.

### Significance and Conclusions

The results suggest that Lmx1a/b regulate mDA axon guidance by controlling Plexin C1 expression. Sema7a, by binding to Plexin C1, have a chemo-repulsive effect on mDA axons. Analysis of Sema7a KO mice suggests that mDA axons from VTA and expressing Plexin C1 are repelled from the dorsal striatal region by Sema7a. This mechanism would explain the segregation of nigrostriatal and mesolimbic dopaminergic axonal pathways.

The main characteristic of PD is the specific loss of mDA neurons from SNpc. The graft of dopaminergic neurons newly generated from stem cells represents a promising therapeutic avenue for PD. However, being able to differentiate stem cells into mDA neurons of the SNpc type represents a real challenge. Data from this study suggest that inactivation of Plexin C1 in newly generated DA neurons from stem cells will dramatically increase the success of a cell replacement therapy for PD. By inactivating Plexin C1, newly generated DA neurons will not be repelled by Sema7a and will likely regenerate a functional network, and thus contribute to the efficiency of cell replacement therapy for PD.

### Materials and Methods

### Animals

Mice Dat-cre; Lmx1aF/+; Lmx1bF/F males, and Lmx1aF/F; Lmx1bF/F females; were obtained from Ang Laboratory. Genotyping was done as previously described (Mishima et al, 2009). Double conditional Dat-cre; Lmx1aF/F; Lmx1bF/F mice were generated by intercrossing Dat-cre; Lmx1aF/+; Lmx1bF/F males, and Lmx1aF/F; Lmx1bF/F mouse lines. Mice Pitx3-GFP (Zhao et al., 2004) were used at E14.5. The embryos were generated, and the day of detection of a copulatory plug was considered E0.5. Used animals were euthanized by ketamine-xylazine injection followed by dislocation. The experiments were performed in accordance with the Canadian Guide for the Care and Use of Laboratory Animals and were approved by the Université Laval Animal Protection Committee.

### Laser Capture Microdissection

Laser capture microdissection (LCM) was applied on thin brain slices (12 μm) for specific isolation of cells with high spatial resolution. By combining rapid TH immunolabeling with LCM, specific cell type were visualized and isolated from surrounding tissue. Microdissected cells are collected in lysis buffer and RNA extracted using picopure RNA isolation kit (Life technologies). Quality and quantity of extracted RNA is then measured using a bioanalyzer (picochip, Illumina). Further analysis of gene expression using mRNA sequencing and rt-qPCR have been performed.

### In Situ Hybridization

Embryos brains were frozen on dry ice. Sections (12 μm) were cut and collected on slides. In situ hybridization (ISH) with digoxigenin-labeled RNA probes was performed as previously described (Conion and Herrman, 1993; Schaeren-Wiemers and Gerfin-Moser, 1993). Plexin C1 mouse antisense RNA probe was used.

### Experimental Procedures

### In Vitro Culture of mDA Neuron Explants

Embryonic ventral midbrain explants dissected from E14.5 Pitx3-GFP embryos (in L15 5% FBS) were grown for 48 h in collagen matrix (Matrigel™, BD) in growth medium (86.8% NDS, 5% P/S, 2% B27, 0.2%, 1% NaPyruvate, 5% FBS) at 37° C., 5% CO2. Then explants were exposed to Sema7a (0.5 ug/ml) for 40 minutes in medium without serum prior fixation (18.5% PFA 20%, 13.3% Sucrose 30%, 68.2% DPBS1X). Some explants were treated before Sema7a application with β1-integrin function blocking antibody for two hours.

### Immunohistochemistry

Embryos were incubated in 4% para-formaldehyde in PBS at 4° C., followed by cryoprotection in 30% sucrose in PBS, before freezing on dry ice. After cryostat sectioning, sections were washed in PBS, then blocked in 1% normal donkey serum (NDS) for at least 30 min. Primary antibodies used in this study were: rabbit anti-TH (Pel-Freez, 1:1000), sheep anti-TH (Millipore, 1:1000), sheep anti-Plexin C1 (R&D systems, 1:150. Secondary antibodies: donkey anti-rabbit Alexa-Fluor-488, donkey anti-sheep Alexa-Fluor-555, were used at 1:200 (Jackson immune); goat anti-rabbit Alexa-Fluor-790 1:1000, donkey anti-sheep Alexa-Fluor-680 1:5000 (Jackson).

### Ex-Vivo Brain Slice Imaging Using Multiphoton Microscopy

The technique of multiphoton microscopy on living brain slice was performed as previously described (Breton-Provencher et al., 2014). Sagittal brain sections from a Pitx3-GFP mouse embryo at E14.5 were used with puff application of Sema7a to create a gradient. Time lapse imaging was performed every 5 min to monitor growth cones movements.

### REFERENCES

1. Breton-Provencher, V.; Coté, D. & Saghatelyan, A. (2014), ‘Activity of the principal cells of the olfactory bulb promotes a structural dynamic on the distal dendrites of immature adult-born granule cells via activation of NMDA receptors.’, J Neurosci 34(5), 1748-1759.
2. Chung, C. Y.; Licznerski, P.; Alavian, K. N.; Simeone, A.; Lin, Z.; Martin, E.; Vance, J. & Isacson, O. (2010), ‘The transcription factor orthodenticle homeobox 2 influences axonal projections and vulnerability of midbrain dopaminergic neurons.’, Brain 133(Pt 7), 2022-2031.
3. Conlon, R. A. & Herrmann, B. G. (1993), ‘Detection of messenger RNA by in situ hybridization to postimplantation embryo whole mounts.’, Methods Enzymol 225, 373-383.
4. Doi, D.; Samata, B.; Katsukawa, M.; Kikuchi, T.; Morizane, A.; Ono, Y.; Sekiguchi, K.; Nakagawa, M.; Parmar, M. & Takahashi, J. (2014), ‘Isolation of human induced pluripotent stem cell-derived dopaminergic progenitors by cell sorting for successful transplantation.’, Stem Cell Reports 2(3), 337-350.
5. Mishima, Y.; Lindgren, A. G.; Chizhikov, V. V.; Johnson, R. L. & Millen, K. J. (2009), ‘Overlapping function of Lmx1a and Lmx1b in anterior hindbrain roof plate formation and cerebellar growth.’, J Neurosci 29(36), 11377-11384.
6. Purro, S. A.; Ciani, L.; Hoyos-Flight, M.; Stamatakou, E.; Siomou, E. & Salinas, P. C. (2008), ‘Wnt regulates axon behavior through changes in microtubule growth directionality: a new role for adenomatous polyposis coli.’, J Neurosci 28(34), 8644-8654.
7. Schaeren-Wiemers, N. & Gerfin-Moser, A. (1993), ‘A single protocol to detect transcripts of various types and expression levels in neural tissue and cultured cells: in situ hybridization using digoxigenin-labelled cRNA probes.’, Histochemistry 100(6), 431-440.
8. Zhao, S.; Maxwell, S.; Jimenez-Beristain, A.; Vives, J.; Kuehner, E.; Zhao, J.; O'Brien, C.; de Felipe, C.; Semina, E. & Li, M. (2004), ‘Generation of embryonic stem cells and transgenic mice expressing green fluorescence protein in midbrain dopaminergic neurons.’, Eur J Neurosci 19(5), 1133-1140.
9. Meyer, A. K.; Maisel, M.; Hermann, A.; Stirl, K. & Storch, A. (2010), ‘Restorative approaches in Parkinson's Disease: which cell type wins the race?’, J Neurol Sci 289(1-2), 93-103.
10. Takahashi, K.; Tanabe, K.; Ohnuki, M.; Narita, M.; Ichisaka, T.; Tomoda, K. & Yamanaka, S. (2007), ‘Induction of pluripotent stem cells from adult human fibroblasts by defined factors.’, Cell 131(5), 861-872.
11. Takahashi, K. & Yamanaka, S. (2006), ‘Induction of pluripotent stem cells from mouse embryonic and adult fibroblast cultures by defined factors.’, Cell 126(4), 663-676.
12. Gao, A.; Peng, Y.; Deng, Y. & Qing, H. (2013), ‘Potential therapeutic applications of differentiated induced pluripotent stem cells (iPSCs) in the treatment of neurodegenerative diseases.’, Neuroscience 228, 47-59.
13. Wernig, M.; Zhao, J.-P.; Pruszak, J.; Hedlund, E.; Fu, D.; Soldner, F.; Broccoli, V.; Constantine-Paton, M.; Isacson, O. & Jaenisch, R. (2008), ‘Neurons derived from reprogrammed fibroblasts functionally integrate into the fetal brain and improve symptoms of rats with Parkinson's disease.’, Proc Natl Acad Sci USA 105(15), 5856-5861.
14. Yu, J.; Vodyanik, M. A.; Smuga-Otto, K.; Antosiewicz-Bourget, J.; Frane, J. L.; Tian,

S.; Nie, J.; Jonsdottir, G. A.; Ruotti, V.; Stewart, R.; Slukvin, I. I. & Thomson, J. A. (2007), ‘Induced pluripotent stem cell lines derived from human somatic cells.’, Science 318(5858), 1917-1920.
15. Okita, K.; Nakagawa, M.; Hyenjong, H.; Ichisaka, T. & Yamanaka, S. (2008),

‘Generation of mouse induced pluripotent stem cells without viral vectors.’, Science 322(5903), 949-953.

1. A method for treating a subject with Parkinson's disease, the method comprising implanting dopaminergic (DA) neurons in which Plexin C1 have been inactivated into the striatum and/or in the midbrain of the subject.

2. The method of claim 1, wherein the DA neurons comprise axons which are not repelled by semaphorin 7a (Sema7A).

3. The method of claim 1, wherein Plexin C1 in the DA neurons is inactivated by:
(a) gene knock-down;
(b) small interfering RNA (siRNA) or short hairpin RNA (shRNA);
(c) gene knock-out;
(d) using a Cre-Lox recombination system;
(e) using a Zinc finger system; or
(f) using a CRISPR/Cas system.

4. The method of claim 3, wherein the CRISPR/Cas system is a CRISPR/Cas9 system.

5. The method of claim 1, wherein the DA neurons are differentiated from stem cells, precursor cells, or progenitor cells in which Plexin C1 has been inactivated.

6. The method of claim 5, wherein the stem cells are embryonic stem cells, neural stem cells (NSCs), multipotent stem cells, or induced pluripotent stem cells (iPSCs).

7. The method of claim 6, wherein the NSCs are fetal NSCs.

8. The method of claim 6, wherein the neural stem cells are adult NSCs.

9. The method of claim 1, wherein the DA neurons are derived from direct conversion of fibroblasts in which Plexin C1 has been inactivated.

10. The method of claim 1, wherein the DA neurons are implanted into the striatum and/or in the midbrain of the subject via local injection.

11. A method for treating a subject with Parkinson's disease, the method comprising administering dopaminergic (DA) neurons in which Plexin C1 have been inactivated and allowing the DA to be implanted into the striatum and/or in the midbrain of the subject.

12. The method of claim 11, wherein the DA neurons comprise axons which are not repelled by semaphorin 7a (Sema7A).

13. The method of claim 11, wherein Plexin C1 in the DA neurons is inactivated by:
(a) gene knock-down;
(b) small interfering RNA (siRNA) or short hairpin RNA (shRNA);
(c) gene knock-out;
(d) using a Cre-Lox recombination system;
(e) using a Zinc finger system; or
(f) using a CRISPR/Cas system.

14. The method of claim 13, wherein the CRISPR/Cas system is a CRISPR/Cas9 system.

15. The method of claim 11, wherein the DA neurons are differentiated from stem cells, precursor cells, or progenitor cells in which Plexin C1 has been inactivated.

16. The method of claim 15, wherein the stem cells are embryonic stem cells, neural stem cells (NSCs), multipotent stem cells, or induced pluripotent stem cells (iPSCs).

17. The method of claim 16, wherein the NSCs are fetal NSCs.

18. The method of claim 16, wherein the neural stem cells are adult NSCs.

19. The method of claim 11, wherein the DA neurons are derived from direct conversion of fibroblasts in which Plexin C1 has been inactivated.

20. The method of claim 11, wherein the DA neurons are administered systemically to the subject prior to implantation into the striatum and/or in the midbrain of the subject.
